(19) United States
(12) Patent Application Publication
(10) Pub. No.: US 2009/0044604 A1
(43) Pub. Date: Feb. 19, 2009

(54) Portable Measuring Facility for Determining A Medically Significant Analyte Concentration

(76) Inventors: Gregor Ocvirk, Mannheim (DE); Peter Kraemer, Deidesheim (DE)

Correspondence Address: BARNES & THORNBURG LLP (Roche), 11 SOUTH MERIDAN STREET, INDIANAPOLIS, IN, 46204, US

(21) Appl. No.: 11/839,240

(22) Filed: Aug. 15, 2007

(51) Int. Cl. G01N 33/49; G01N 33/487

(52) U.S. Cl. 73/61.41

(57) ABSTRACT

A portable measuring facility for determining the concentration of an analyte in a living body may comprise a sensor configured to determine the concentration of an analyte in a liquid, and a liquid conveying facility. The liquid conveying facility may have a liquid line configured to be connected to a probe that is implantable into tissue of the living body, a pump configured to pump the liquid from the probe through the liquid line to the sensor, and a degassing facility configured to degas the liquid pumped from the probe through the liquid line to the sensor.

### FIELD OF THE INVENTION

The invention relates generally to a portable measuring facility for determining the concentration of an analyte in a living body, and more specifically to such measuring facilities that may comprise a sensor for determining the concentration of an analyte that is present in a liquid, and a liquid conveying facility having a liquid line for connection to a probe that can be implanted into body tissue, and a pump for pumping liquid from the probe through the liquid line to the sensor.

### BACKGROUND

Measuring facilities of this type are known, for example in the form of so-called micro-dialysis systems, an example of which is described in US 2002/0082490 A1. In a micro-dialysis system, a perfusion liquid is pumped through a micro-dialysis probe that is implanted in body tissue such that analyte molecules from body tissue and/or body fluid surrounding the probe diffuse into the perfusion liquid. In an ideal case, the analyte concentration becoming established in the perfusion liquid is identical to that in the body tissue surrounding the probe. Subsequently, the analyte concentration in the perfusion liquid is measured with a sensor of the micro-dialysis system in order to determine the concentration of the analyte in a human or animal body fluid.

One application of micro-dialysis systems is the measurement of the glucose concentration for diabetes treatment. The measuring facilities used for this purpose should ideally be small and lightweight, since they are taken along by diabetics at all times. However, as a matter of principle, there is no need in the continuous measurement of the glucose content using a portable measuring facility to pump a perfusion solution through a micro-dialysis probe. It is also feasible to continuously remove a small amount of body fluid from body tissue using a probe and to determine the concentration in the body fluid directly by means of a sensor.

On the one hand, portable measuring facilities of the type specified above should be as small and lightweight as possible, in order to allow a patient to take them along at all times, and, on the other hand, they should determine a medically-significant analyte concentration both reliably and at high precision.

It is desirable to devise a way of improving the measuring accuracy of a portable measuring facility of the type specified above without increasing the size and weight of the measuring facility to a significant extent.

### SUMMARY

A portable measuring facility of the type specified above, includes a liquid conveying facility that comprises a degassing facility for degassing the liquid that is to be guided from the probe through the liquid line to the sensor.

It has been determined that that the measuring accuracy of such measuring facilities are negatively affected by variations of the gas content of the liquid to be analyzed, in particular by gas bubbles being present therein. Consequently, the measuring accuracy can be increased significantly by degassing the liquid to be analyzed. It is not absolutely necessary to completely degas the liquid to be tested, since even just the removal of gas bubbles allows both the precision and the reliability of analyte concentration measuring results to be improved substantially.

In order to be able to determine a medically-significant analyte concentration using portable measuring facilities, various consumables aside from the actual measuring facility are required, for example probes, which, according to their intended purpose, are implanted in body tissue, and tubes for connecting a probe to the liquid line of the measuring facility. Consumables of this type and a corresponding measuring facility form a portable measuring system for determining an analyte concentration. For this reason, another aspect of this disclosure relates to a portable measuring system for determining a medically-significant analyte concentration using a measuring facility that comprises a sensor and a liquid conveying facility, and a probe that can be implanted in body tissue which, in operation, is connected to the liquid conveying facility.

The degassing facility of a measuring facility illustratively preferably comprises an aspiration line that is used, in operation, to apply a negative pressure to a gas-permeable surface of the liquid line. For example, a section of the liquid line can be provided in the form of a gas-permeable hollow fiber that is arranged within a degassing chamber to which the aspiration line is attached. In this context, it is particularly favorable for the liquid line to comprise a section that is provided in the form of a tube on which the pump acts in a peristaltic fashion in order to generate a conveying pressure, and for the aspiration line to also comprise a section that is provided in the form of a tube on which the pump acts in a peristaltic fashion in order to generate a negative pressure. By this means, a single pump, which is needed anyway for a liquid conveying facility, can be used to generate both the requisite conveying pressure and the negative pressure, and a degassing of the liquid to be analyzed can be effected with a compact device.

Peristaltic pumps are known from other areas of engineering and are described, for example, in WO 2004/109109 A1. A peristaltic pump can be used to implement both an even conveying performance, which is important for a precise analysis, and also a compact degassing facility. Therefore, another aspect of this disclosure relates to a liquid conveying facility for degassing of liquids, comprising a liquid line for conveying the liquid, a pump for generating a conveying pressure in the liquid line, and an aspiration line that is used, in operation, to apply a negative pressure to a gas-permeable surface of the liquid line, whereby the pump is a peristaltic pump, the liquid line comprises a section that is provided in the form of a tube on which the pump, in operation, acts in a peristaltic fashion in order to generate the conveying pressure, and the aspiration line comprises a section that is provided in the form of a tube on which the pump, in operation, acts in a peristaltic fashion in order to generate the negative pressure.

### BRIEF DESCRIPTION OF THE DRAWINGS

Further details of the invention are illustrated in the following by means of exemplary embodiments and referring to the appended drawings, whereby equal and corresponding parts are identified therein by identical reference numbers. The features of the exemplary embodiments described in the following can be made the subject of claims individually or in combination. In the figures:

FIG. 1 shows a schematic view of an exemplary embodiment of a measuring facility;

FIG. 2 shows a schematic view of another exemplary embodiment of a measuring facility;

FIG. 3 shows a schematic view of another exemplary embodiment of a measuring facility; and

FIG. 4 shows a schematic view of another exemplary embodiment of a measuring facility.

### DESCRIPTION OF THE ILLUSTRATIVE EMBODIMENTS

For the purposes of promoting an understanding of the principles of the invention, reference will now be made to a number of illustrative embodiments shown in the attached drawings and specific language will be used to describe the same.

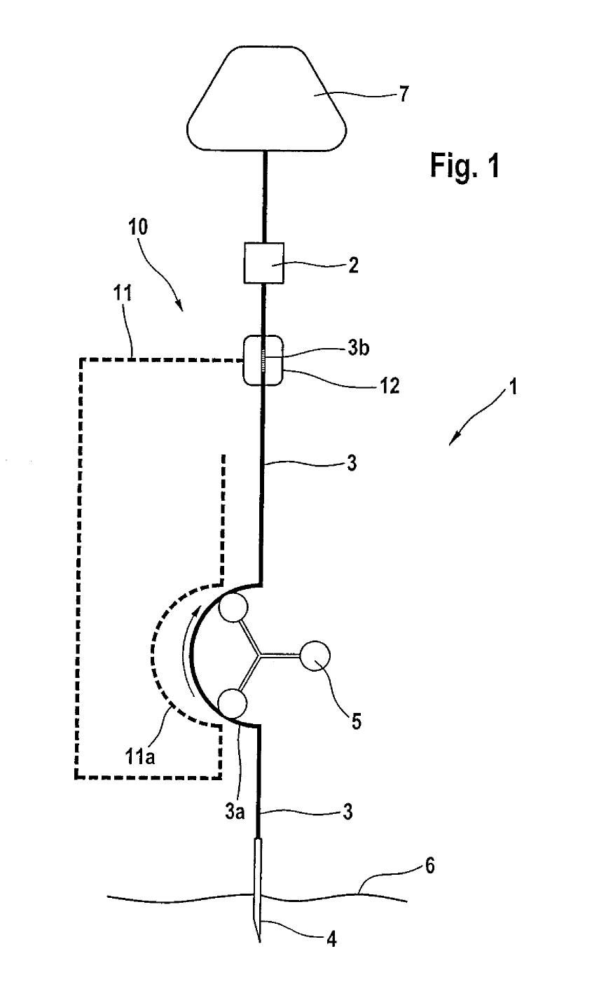

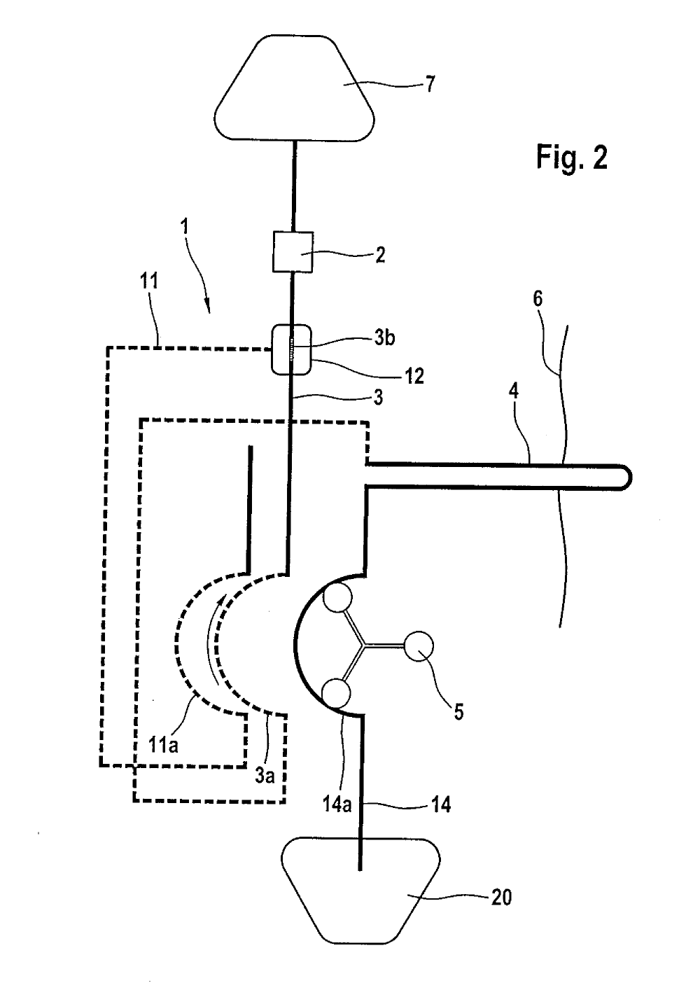

FIG. 1 shows a schematic view of an exemplary embodiment of a measuring facility 1 for determining a concentration of an analyte in a living body. The analyte of the exemplary embodiment shown is glucose. The measuring facility 1 comprises a sensor 2 for determining the concentration of an analyte in a liquid and a liquid conveying facility having a liquid line 3 for connection to a probe 4 that can be implanted into body tissue, and a pump 5 in order to pump liquid from the probe 4 through the liquid line 3 to the glucose sensor 2. The measuring facility 1 and the probe 4, shown implanted into a human body 6 in FIG. 2, together form a portable measuring system that is independent of mains supply. The sensor 2 is an electrochemical sensor. The sensor 2 is provided in the form of a flow-through sensor.

In the exemplary embodiment shown in FIG. 1, the pump 5 is used to pump a body fluid, for example blood, from probe 4 through the liquid line 3 to the sensor 2. The tested liquid is then guided from the sensor 2 to a waste container 7, in which it is stored until disposal. In order to increase the measuring accuracy of the sensor 2, the liquid conveying facility contains a degassing facility 10 for degassing the liquid that is to be guided from the probe 4 through the liquid line 3 to the sensor 2. The degassing facility 10 is arranged downstream from the pump 5.

The degassing facility 10 comprises an aspiration line 11 that is shown by dashed lines in FIG. 1 and, in operation, is used to apply a negative pressure to a gas-permeable surface of the liquid line 3. The gas-permeable surface of the liquid line is arranged within a degassing chamber 12 to which the aspiration line 11 is connected. The gas-permeable surface of the liquid line 3 is provided by a hollow fiber that forms a section of the liquid line that is arranged within the degassing chamber 12. Suitable hollow fibers having a gas-permeable, but liquid-tight, surface are marketed by Membrana under the names, Oxyphan or Oxyplus. Suitable materials for hollow fibers of this type are, in particular, hydrophobic chain molecules, such as polypropylene and polymethylpentene.

The pump 5 is a peristaltic pump that is used to generate both the conveying pressure of the liquid line 3 and the negative pressure of the aspiration line 11. The liquid line 3 comprises a section 3a that is provided in the form of a tube on which the pump 5, in operation, acts in a peristaltic fashion in order to generate the conveying pressure. The aspiration line 11 comprises a section 11a that is provided in the form of a tube on which the pump 5, in operation, acts in a peristaltic fashion in order to generate the negative pressure.

Peristaltic pumps essentially consist of a tube bed as a support for the tube and at least one element that compresses a partial area of the tube. The pumping action is generated by the compressing element being moved in longitudinal direction with respect to the tube. Known compressing elements include plungers, fingers or rollers on a rotor. Three rollers that are being moved along the tube in the direction of the arrows are depicted in the exemplary embodiment shown. The tube sections 3a, 11a of the liquid line 3 and aspiration line 11, respectively, are arranged side by side on a tube bed such that the compressing elements of the peristaltic pump each act simultaneously on both tubes. In this context, it is favorable to use identical tubes, in particular tubes made of the same material, for example PVC, and having identical external diameters, for the corresponding sections 3a, 11a of the liquid line 3 and aspiration line 11, respectively.

It is favorable for the first section 3a of the liquid line 3, that is provided in the form of a tube on which the pump 5 acts in a peristaltic fashion in order to generate the conveying pressure, to have a thicker wall than a second liquid line section 3b that is arranged within the degassing chamber 12. By this means, the first section 3a of the liquid line 3 on which the pump 5 acts in a peristaltic fashion in order to generate the conveying pressure can be provided to be more resistant such that it can resist the peristaltic impact of the pump 5 for long periods of time without showing symptoms of wear. In contrast, the second liquid line section 3b that is arranged within the degassing chamber 12 is not exposed to stress of this type such that it can be provided particularly thin-walled in order to provide the gas-permeable surface. The two sections, 3a, 3b, are connected to each other in a substance-to-substance bonding fashion, for example by gluing. With regard to the dimensioning of the hollow space of the degassing chamber 12 and of the second section 3b of the liquid line 3 extending therein, it should be ensured, on the one hand, that the hollow space of the degassing chamber 12 is sufficiently long in order to permit sufficient transfer of gas for the removal of gas bubbles, if any, and, on the other hand, is as short as possible to allow the requisite negative pressure in the degassing chamber 12 to be generated as quickly as possible at the time of start-up. The optimal length of the degassing chamber 12 therefore depends on the flow velocity of the liquid in the liquid line 3 and on the internal diameter of the tube section 11. Favorable are conveying rates of the liquid conveying facility from 0.1 μl/min to 1 μl/min, in particular 0.2 μl/min to 0.4 μl/min. Moreover, it must be ensured that the volume of the hollow space of the degassing chamber 12 surrounding the section 3b of the liquid line 3 is dimensioned sufficiently large for the relative moisture content of the hollow space to be kept low in operation and to allow the requisite negative pressure to be maintained.

The wall thickness of the second liquid line section 3b that is arranged within the degassing chamber 12 illustratively is less than 0.2 mm, in particular less than 0.1 mm. The wall thickness of the first section 3a of the liquid line 3 that is provided in the form of a tube and on which the pump 5 acts in a peristaltic fashion in order to generate the conveying pressure illustratively is at least 0.5 mm, in particular the wall thickness is 0.7 mm to 2 mm. The internal diameter of the tube on which the pump 5 acts in a peristaltic fashion illustratively is 0.5 mm to 0.3 mm. The internal diameter of the second section of the liquid line that is arranged within the degassing chamber 12 illustratively is of 0.1 mm to 0.4 mm, in particular 0.2 mm to 0.4 mm. The length of the degassing chamber 12 illustratively corresponds to 5- to 50-fold the internal diameter of the section of the liquid line 3 that extends within the degassing chamber 12. It is favorable for the volume of the hollow space of the degassing chamber 12 to be 1.2- to 1200-fold the volume taken up within the hollow space by the second section 3b of the liquid line 3 that is arranged therein. Illustratively, the degassing chamber 12 is provided in the form of a T-piece made of plastic such that the liquid line 3 can be guided through the degassing chamber 12 with little effort and the aspiration line 11 can be connected to the degassing chamber 12.

To allow the measuring facility shown to be taken along by a user at all times, it is operable independent of mains supply and therefore has a reception compartment (not shown) for batteries. The mass of the measuring facility is less than 1 kg such that its weight is only a small load for a user.

FIG. 2 schematically shows another exemplary embodiment of a measuring system that comprises a measuring facility 1 and a probe 4 that is shown while it is implanted in a living body in FIG. 2. Whereas, in the exemplary embodiment illustrated by means of FIG. 1, a body fluid sample 1 is removed using the probe 4 and analyzed by means of the sensor 2, a perfusion liquid is guided through the probe 4 that is provided in the form of a micro-dialysis probe in the measuring system shown in FIG. 2. The micro-dialysis probe 4 has a dialysis membrane through which analyte molecules from body fluid surrounding the probe 4 can diffuse into the perfusion liquid such that the perfusion liquid can subsequently be guided through the liquid line 3 to the sensor 2 for determining the analyte concentration. The measuring system shown therefore is a micro-dialysis system.

The measuring facility 1 shown in FIG. 2 has a perfusion liquid line 14 that serves to guide perfusion liquid from a perfusion liquid reservoir 20 into the micro-dialysis probe 4. A section 14a of the perfusion liquid line 14 is provided in the form of a tube on which the pump 5 acts in a peristaltic fashion in order to generate a conveying pressure. This tube section 14a is arranged in the tube bed of the pump 5 side by side with the tubes 3a, 11a of the aspiration line 11 and liquid line 3 such that the compressing elements of the peristaltic pump 5 act simultaneously on all 3 tubes 3a, 11a, 14a.

Suitable probes 4 and further details of the micro-dialysis system are disclosed in US 2002/0082490 A1, the disclosure of which is incorporated herein by reference. One difference between the measuring system shown in FIG. 2 and the micro-dialysis system known from US 2002/0082490 A1 is that a liquid conveying facility having a degassing facility 11, 12 was added in the exemplary embodiment shown in order to increase the reliability and accuracy of the analyte concentration information obtained with the sensor 2.

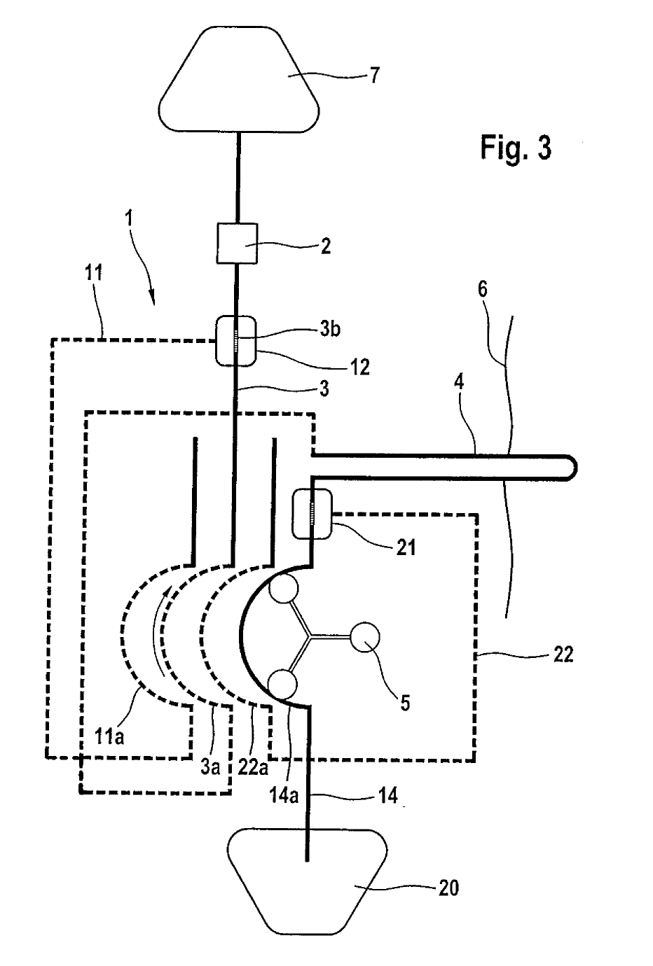

FIG. 3 schematically shows another exemplary embodiment of a micro-dialysis system. The micro-dialysis system shown in FIG. 3 differs from the micro-dialysis system shown in FIG. 2 in that a second degassing facility having a second degassing chamber 21 for degassing the perfusion liquid to be guided into the probe 4 is added. In the second exemplary embodiment shown, the perfusion line 14 extends through the second degassing chamber 21 to which a second negative pressure line 22 is connected. The negative pressure line 22 also has a section 22a that is provided in the form of a tube on which the peristaltic pump 5 acts in a peristaltic fashion in order to generate the negative pressure. Like the corresponding second section 3a of the liquid line 3 that extends in the first degassing chamber 12, the section 14a of the perfusion liquid line 14 that extends in the second degassing chamber 21 has a gas-permeable surface and can be provided, for example, in the form of a hollow fiber.

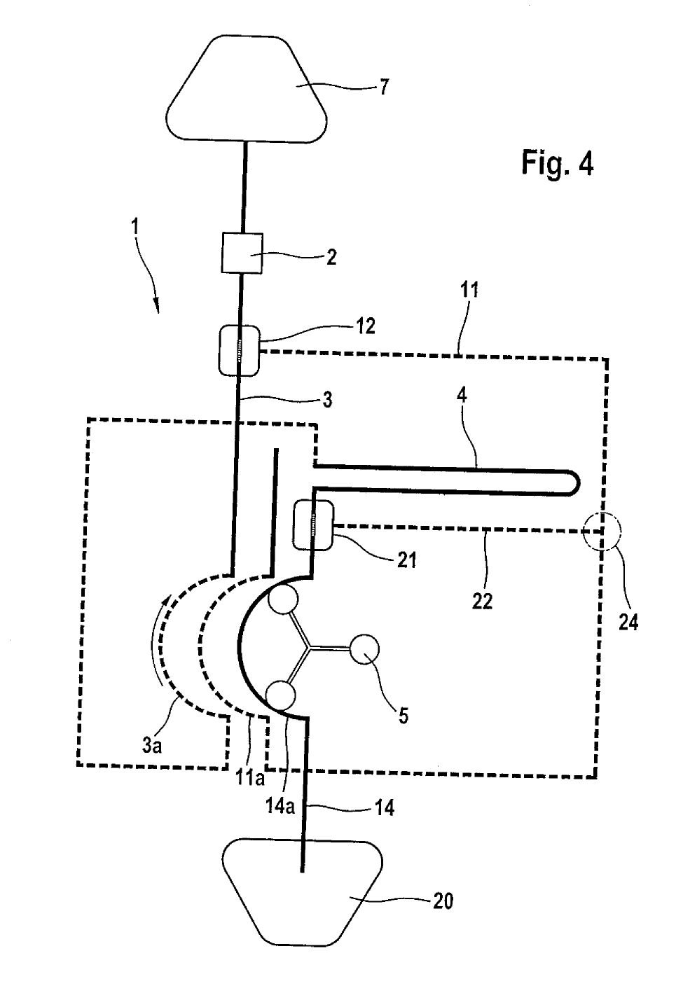

FIG. 4 schematically shows another exemplary embodiment of a micro-dialysis system. The micro-dialysis system shown in FIG. 4 differs from the micro-dialysis system shown in FIG. 3 in that the negative pressure line 11 and the negative pressure line 22 are connected to each other by means of a T-piece 24 such that only one section 11a that is provided in the form of a tube on which the peristaltic pump 5 acts in a peristaltic fashion in order to generate the negative pressure is required. Unlike in FIG. 3, this provides the opportunity to reduce the required momentum of the peristaltic pump 5 and thus its current consumption.

While the invention has been illustrated and described in detail in the foregoing drawings and description, the same is to be considered as illustrative and not restrictive in character, it being understood that only illustrative embodiments thereof have been shown and described and that all changes and modifications that come within the spirit of the invention are desired to be protected.

What is claimed is:

1. A portable measuring facility for determining the concentration of an analyte in a living body, comprising
a sensor configured to determine the concentration of an analyte in a liquid, and
a liquid conveying facility having a liquid line configured to be connected to a probe that is implantable into tissue of the living body, a pump configured to pump the liquid from the probe through the liquid line to the sensor, and a degassing facility configured to degas the liquid pumped from the probe through the liquid line to the sensor.

2. The measuring facility of claim 1 wherein a mass of the measuring facility is less than 1 kg.

3. The measuring facility of claim 1 wherein the measuring facility is a micro-dialysis facility having a perfusion liquid line that guides perfusion liquid from a perfusion liquid reservoir into the probe.

4. The measuring facility of claim 3 wherein the probe is a micro-dialysis probe comprising a dialysis membrane through which molecules of the analyte from body fluid surrounding the probe can diffuse into the perfusion liquid,
and wherein the liquid pumped from the probe through the liquid line to the sensor is the perfusion liquid with the molecules of the analyte diffused therein.

5. The measuring facility of claim 4 wherein the degassing facility comprises an aspiration line configured to apply a negative pressure to a gas-permeable surface of the liquid line.

6. The measuring facility of claim 5 further comprising a degassing chamber connected to the aspiration line,
wherein the gas-permeable surface of the liquid line is arranged within the degassing chamber.

7. The measuring facility of claim 6 wherein a length of the degassing chamber is 5- to 20-fold an internal diameter of a section of the liquid line that is arranged within the degassing chamber.

8. The measuring facility of claim 5 wherein the pump is a peristaltic pump,
wherein a section of the liquid line comprises a first tube on which the pump acts in peristaltic fashion to generate a conveying pressure that pumps the liquid from the probe through the liquid line to the sensor,
and wherein a section of the aspiration line comprises a second tube on which the pump acts in peristaltic fashion to generate the negative pressure.

9. The measuring facility of claim 8 further comprising a degassing chamber connected to the aspiration line,
wherein the gas-permeable surface of the liquid line is arranged within the degassing chamber.

10. The measuring facility of claim 8 wherein a wall thickness of the first tube is larger than that of a section of the liquid line having the gas-permeable surface.

11. The measuring facility of claim 10 wherein the section of the liquid line having the gas-permeable surface is a hollow fiber.

12. The measuring facility of claim 10 wherein a length of the degassing chamber is 5- to 20-fold an internal diameter of a section of the liquid line that is arranged within the degassing chamber.

13. The measuring facility of claim 1 wherein the degassing facility is arranged downstream from the pump.

14. A portable measuring facility for determining the concentration of an analyte in a living body, comprising
a sensor configured to determine the concentration of an analyte that is present in a liquid, and
a liquid conveying facility having a liquid line configured to be connected to a probe that is implantable into tissue of the living body, a pump configured to pump the liquid from the probe through the liquid line to the sensor, and a degassing facility having an aspiration line configured to degas the liquid pumped from the probe through the liquid line to the sensor by applying a negative pressure to a gas-permeable surface of the liquid line.

15. The measuring facility of claim 14 further comprising a degassing chamber connected to the aspiration line,
wherein the gas-permeable surface of the liquid line is arranged within the degassing chamber.

16. The measuring facility of claim 15 wherein a length of the degassing chamber is 5- to 20-fold an internal diameter of a section of the liquid line that is arranged within the degassing chamber.

17. The measuring facility of claim 14 wherein the pump is a peristaltic pump,
wherein a section of the liquid line comprises a first tube on which the pump acts in peristaltic fashion to generate a conveying pressure that pumps the liquid from the probe through the liquid line to the sensor,
and wherein a section of the aspiration line comprises a second tube on which the pump acts in peristaltic fashion to generate the negative pressure.

18. The measuring facility of claim 17 further comprising a degassing chamber connected to the aspiration line,
wherein the gas-permeable surface of the liquid line is arranged within the degassing chamber.

19. The measuring facility of claim 17 wherein a wall thickness of the first tube is larger than that of a section of the liquid line having the gas-permeable surface.

20. The measuring facility of claim 19 wherein the section of the liquid line having the gas-permeable surface is a hollow fiber.

21. The measuring facility of claim 19 wherein a length of the degassing chamber is 5- to 20-fold an internal diameter of a section of the liquid line that is arranged within the degassing chamber.

22. The measuring facility of claim 14 wherein the degassing facility is arranged downstream from the pump.

23. The measuring facility of claim 14 wherein a mass of the measuring facility is less than 1 kg.

24. A portable measuring facility for determining the concentration of an analyte in a living body, comprising
a sensor configured to determine a concentration of an analyte in a liquid,
a probe configured to be implanted into tissue of the living body,
a peristaltic pump,
a liquid line having a section defining a first tube on which the pump acts in peristaltic fashion to generate a conveying pressure in the liquid line to convey the liquid from the probe through the liquid line to the sensor, and
an aspiration line having a section comprising a second tube on which the pump acts in peristaltic fashion to generate a negative pressure that is applied to a gas-permeable surface of the liquid line.

25. The measuring facility of claim 24 further comprising a degassing chamber connected to the aspiration line,
wherein the gas-permeable surface of the liquid line is arranged within the degassing chamber.

26. The measuring facility of claim 25 wherein the degassing facility is arranged downstream from the pump.

27. The measuring facility of claim 25 wherein a mass of the measuring facility is less than 1 kg.

28. The measuring facility of claim 25 wherein the measuring facility is a micro-dialysis facility having a perfusion liquid line that guides perfusion liquid from a perfusion liquid reservoir into the probe,
wherein the probe is a micro-dialysis probe comprising a dialysis membrane through which molecules of the analyte from body fluid surrounding the probe can diffuse into the perfusion liquid,
and wherein the liquid being conveyed is the perfusion liquid with the molecules of the analyte diffused therein.
